(19) United States
(12) Patent Application Publication
(10) Pub. No.: US 2004/0014101 A1
(43) Pub. Date: Jan. 22, 2004

(54) Separating and/or identifying polymorphic nucleic acids using universal bases

(75) Inventors: Xiangjun Liu, Menomonee Falls, WI (US); Lu Wang, Shorewood, WI (US); Daniel S. Ramon, Brown Deer, WI (US)

Correspondence Address: FOLEY & LARDNER, 150 EAST GILMAN STREET, P.O. BOX 1497, MADISON, WI, 53701-1497, US

(73) Assignee: Pel-Freez Clinical Systems, Inc.

(21) Appl. No.: 10/429,912

(22) Filed: May 5, 2003

Related U.S. Application Data
(60) Provisional application No. 60/377,507, filed on May 3, 2002.

(51) Int. Cl. C12Q 1/68

(52) U.S. Cl. 435/6

(57) ABSTRACT

The present invention provides methods for analyzing polymorphic nucleic acids using duplex separation and/or identification techniques. In the present methods one of the nucleic acids has a sequence which includes universal bases that correspond in position to one or more of the polymorphic positions of the polymorphic nucleic acid. The nucleic acid including the universal bases can either represent the polymorphic nucleic acid and hybridize with a reference strand, or act as a reference strand and hybridize with the polymorphic nucleic acid directly, to form a duplex. Separation and/or identification of the duplex from other duplexes and materials can then be performed.

### CLAIM FOR PRIORITY

The present application claims priority to U.S. Patent Application Serial No. 60/377,507, the entire contents of which are hereby incorporated by reference.

### FIELD OF INVENTION

The present invention relates to the separation and/or identification of polymorphic nucleic acids using hybridization techniques. More specifically, the present invention relates to separating and identifying polymorphic nucleic acids using nucleic acids that have one or more universal bases.

### BACKGROUND OF THE INVENTION

The detection of specific nucleic acids is an important tool for diagnostic medicine and molecular biology research. Nucleic acid identification currently plays an important role in identifying infectious organisms such as bacteria and viruses, in probing the expression of normal genes and identifying mutant genes such as oncogenes, in typing tissue for compatibility preceding tissue transplantation, in matching tissue or blood samples for forensic medicine and for exploring homology among genes from different species. One method used to type and identify tissues involves formation of a duplex between a nucleic acid of a sample with a reference nucleic acid and subsequent separation of the duplex from others in the sample, such as through gel electrophoresis.

One major focus of tissue typing, paternity testing and disease association has been on the human leukocyte antigen (HLA) gene. The HLA alleles are the most diverse antigenic system in the human genome and encode literally hundreds of alleles that fall into several distinct subgroups or subfamilies. However, standard techniques for DNA typing have often proven inadequate in resolving many of these important alleles. Not only are techniques that are capable of unlocking this genetic diversity rare, such as sequencing analysis, they are also expensive and time consuming.

Thus there remains a continuing need for improving the efficiency and accuracy of separating and identifying polymorphic nucleic acids.

### SUMMARY OF THE INVENTION

In one aspect, the present methods provide for separating and/or identifying a nucleic acid molecule. This embodiment involves hybridizing a first nucleic acid with a reference, or test, nucleic acid to form a test duplex. The first nucleic acid has a sequence that matches, or is complementary to, a nucleic acid having a plurality of polymorphic positions where one or more of the polymorphic positions, or the positions complementary thereto, are replaced with a universal base. In some embodiments, the sequence or identity of the first nucleic acid or reference nucleic acid will be known or predetermined. Test duplexes are then separated. In some embodiments, the test duplex is detected. The identity of one or more of the nucleic acids making up the test duplex can then be determined.

In some embodiments, the test duplex is separated from one or more control duplexes run in the same separation and the positions to which the duplexes are detected. The position to which the test duplex migrates is determined by comparing the migration positions of the duplexes and the identity of the first nucleic acid, reference nucleic acid or both can then be determined based on the position to which the test duplex migrates.

Another method for accomplishing the separation and/or identification of a nucleic acid involves hybridizing a polymorphic nucleic acid having one or more polymorphic positions with a reference nucleic acid that is substantially complementary to the first nucleic acid to form a test duplex. In the reference nucleic acid one or more positions complementary to the one or more polymorphic positions of the first nucleic acid are replaced with a universal base. In some embodiments, the sequence or identity of the reference nucleic acid will be known or predetermined. Separation of the test duplex can then performed. In some embodiments, the presence or absence of the test duplex is detected. The polymorphic nucleic acid can also be identified. In some embodiments, the polymorphic nucleic acid will be identified, simultaneous with, or after the separation and/or detection of the test duplex.

In some of the above embodiments, the test duplex will be denatured into single nucleic acid strands after the separation step prior to detection and/or identification. In some embodiments, the test duplex will be separated under denaturing conditions.

In some embodiments, one or more control duplexes can be performed in the same separation and their positions are detected. Identification of the nucleic first nucleic acids can be based on the position to which the duplexes migrate. In some of these methods, the polymorphic nucleic acid does not form a stable duplex with the reference nucleic acid in the absence of the nucleic acid having universal bases.

In some of these methods the reference nucleic acid has a sufficient amount of positions complementary to the one or more polymorphic positions of the polymorphic nucleic acid replaced with universal bases in order to stabilize the test duplex relative to a test duplex with a control nucleic acid having the same sequence but no universal nucleic bases. Additionally, a sufficient amount of the polymorphic positions, or positions complementary thereto, are not replaced with universal bases such that all alleles which have different polymorphic sequences within the primer annealing region can be identified utilizing the same reference nucleic acid. In yet others of these methods, the reference nucleic acid is capable of specifically identifying all of the HLA alleles of an HLA gene subfamily but not the HLA alleles of related HLA gene subfamilies

In the above embodiments the methods can be repeated as desired, using different first nucleic acids, reference nucleic acids, or polymorphic nucleic acids. In some of the above embodiments, the nucleic acids can be produced through primer extension. In another embodiment, all of the different first nucleic acids can have at least one universal base in a polymorphic position, or position complementary thereto, which is common to all of the first nucleic acids.

One aspect of the invention provides a method for identifying a nucleic acid molecule, comprising some or all of the steps of:

(a) hybridizing a first nucleic acid whose sequence matches, or is complementary to, a nucleic acid having a plurality of polymorphic positions with a reference nucleic acid whose sequence is substantially complementary to the first nucleic acid to form a test duplex, wherein one or more of the polymorphic positions, or the positions complementary thereto, of the first nucleic acid are replaced with a universal base; and

(b) separating the test duplex.

Another aspect of this method further involves:

(c) detecting the test duplex; and/or

(d) identifying the first nucleic acid.

In one aspect the first nucleic acid is produced by:

(i) hybridizing the polymorphic nucleic acid having a plurality of polymorphic positions or the nucleic acid complementary thereto with a complementary nucleic acid primer that has at least one universal base complementary to at least one polymorphic position of the polymorphic nucleic acid; and

(ii) elongating the nucleic acid primer.

In some aspects the polymorphic nucleic acid is an HLA allele, and in particular HLA-DRB1.

In other aspects the methods further comprising repeating the above steps one or more times using different first nucleic acids in each repeat with the same reference nucleic acid or different reference nucleic acids.

Another aspect of the methods provides that all of the different first nucleic acids have at least one universal base in a polymorphic position, or position complementary thereto, which is common to all of the first nucleic acids.

In yet other aspects the universal base is 2′-deoxyinosine, 3-nitropyrrole (3-nitropyrrole 2′-deoxynucleoside), 5-nitroindole (5-nitroindole 2′-deoxynucleoside), 4-nitroindole (4-nitroindole 2′-deoxynucleoside), 6-nitroindole (6-nitroindole 2′-deoxynucleoside), 2′-deoxynebularine, N6-methoxy-2,6-diaminopurine, or 6H,8H-3,4-dihydropyrimido[4,5-c][1,2]oxazin-7-one.

In still other aspects the first nucleic acid, reference nucleic acid or both are labeled, for example at their 5′ or 3′ end.

Another aspect provides a method for identifying a nucleic acid molecule, comprising:

(a) hybridizing a polymorphic nucleic acid having one or more polymorphic positions with a reference nucleic acid that is substantially complementary to the first nucleic acid to form a test duplex, wherein one or more positions of the reference nucleic acid complementary to the one or more polymorphic position are replaced with a universal base;

(b) separating the test duplex.

This method can further involve:

(c) detecting the test duplex; and/or

(d) identifying the polymorphic nucleic acid based. The polymorphic nucleic acid can be identified based on the position to which the test duplex migrates compared to the position to which the one or more control duplexes migrate.

In some aspects the polymorphic nucleic acid does not form a stable duplex with the reference nucleic acid in the absence of the nucleic acid having universal bases. In more aspects the first nucleic acid, reference nucleic acid or polymorphic nucleic acid is a primer, which can be elongated prior to (b).

In still additional aspects the reference nucleic acid has a sufficient amount of the positions complementary to the one or more polymorphic positions of the polymorphic nucleic acid replaced with universal bases in order to stabilize the test duplex relative to a test duplex with a control nucleic acid having the same sequence that does not have universal nucleic acids and further wherein a sufficient amount of the polymorphic positions, or positions complementary thereto, are not replaced with universal bases such that all alleles which have different polymorphic sequences within the primer annealing region can be identified utilizing the same reference nucleic acid.

In other aspects the allele of the gene subfamily is an HLA allele, and in some of these the reference nucleic acid is capable of specifically identifying all of the HLA alleles of an HLA gene subfamily but not the HLA alleles of related HLA gene subfamilies, such as HLA-DRB-1.

In still other aspects the methods further comprise repeating (a)-(d) are repeated one or more times wherein different polymorphic nucleic acids or different reference nucleic acids are used in each repeat.

An aspect of the invention also provides a modified nucleic acid comprising the sequence set forth in SEQ ID NO: 1 (TTGNNGCAGGTTAANNNTGAG), SEQ ID NO: 2 (GNCCCCNCAGCACGTTTCCTGNN GCAGGTTAANNNTGAGTGTCATTTC), SEQ ID NO: 3 (GNCCCCNCAGCACGTTTCNTGNNGNANNNTANNNNNNANTGTNANTTCTT CAAT), SEQ ID NO: 4 (GNCCCCNCAGCACGTTTCNTGNNGNANNNTANNNNTNAG TGTNATTTCTTCAAT) or a nucleic acid having a sequence complementary to SEQ ID NO: 1, SEQ ID NO: 2, SEQ ID NO: 3 or SEQ ID NO: 4, wherein N denotes a universal base that is capable of hybridizing to any other nucleic acid residue.

In some aspects the universal base is 2′-deoxyinosine, 3-nitropyrrole (3-nitropyrrole 2′-deoxynucleoside), 5-nitroindole (5-nitroindole 2′-deoxynucleoside), 4-nitroindole (4-nitroindole 2′-deoxynucleoside), 6-nitroindole (6-nitroindole 2′-deoxynucleoside), 2′-deoxynebularine, N6-methoxy-2,6-diaminopurine, or 6H,8H-3,4-dihydropyrimido[4,5-c][1,2]oxazin-7-one.

Other aspects provide a kit for identifying a nucleic acid molecule comprising: (i) instructions for carrying out the present methods and one or more: (ii) reagents, (iii) primers, (iv) solid supports (v) enzymes or (vi) pieces of lab equipment.

Any aspect disclosed herein can be used with any and all appropriate aspects of the invention.

### BRIEF DESCRIPTION OF THE DRAWINGS

FIG. 1 illustrates a primer having universal bases and the HLA alleles which the primer can amplify.

### DETAILED DESCRIPTION OF PREFERRED EMBODIMENTS

The present methods use nucleic acids that have universal bases to enhance the separation and improve the identification of polymorphic nucleic acids by hybridization techniques. These techniques take advantage of the fact that nucleic acid interactions strongly favor hybridization of adenine with thymine (or uracil in RNA) on the one hand and guanine with cytosine on the other. Mismatches in base pairing or unmatched bases result in destabilization of the nucleic acid duplex due to unfavorable energetic constraints. This instability is exploited in separating nucleic acids because duplexes which have mismatched or unmatched base pairs (heteroduplexes) behave differently not only from each other, but also from perfectly matched duplexes (homoduplexes). For example, in separation techniques, such as chromatography, heteroduplexes migrate more slowly than homoduplexes and differently than heteroduplexes that have different combinations of mismatches and/or unmatched bases. Depending on the number and position of mismatched/unpaired bases, differences as little as one base mismatch/nonpair can be detected upon separation.

Often, in duplex separation techniques nucleic acids are obtained from an individual that has a nucleic acid of interest and amplified using conventional techniques, such as PCR. Once increased to a desired amount, the amplified nucleic acid is denatured into single strands and mixed together with generally complementary single-strand reference nucleic acids and annealed thus forming homoduplexes and/or heteroduplexes. Often the sequence or identity of the reference nucleic acids are know or predetermined. In order to improve the method, one of the strands, either the sense strand or antisense strand, from the individual can be discarded or labeled prior to annealing with the reference strand. Removal of the unwanted strand can be achieved easily through methods well known in the art. Such methods including labeling one of the strands, such as with a high molecular weight moiety, or immobilizing one of the strands on a solid support via a linker, such as an antibody or biotin, denaturing the strands and washing the unbound strand from the mixture. The desired strand, which can be the bound or unbound strand, is then collected and used to form the duplex with the reference strand.

After annealing the reference strand with the nucleic acid of interest, the duplex is then separated from other components in the mixture, including hetero- and homoduplexes, using well-known separation techniques, such as gel electrophoresis, high performance (pressure) liquid chromatography (HPLC), denaturing high performance (pressure) liquid chromatography (DHPLC) and the like based on the duplex migration principles discussed above.

During separation, the test duplex can be run with one or more reference markers. Examples of reference markers include homoduplexes containing the nucleic acid of interest, one or more markers having a known molecular weight or migration pattern and one or more control heteroduplexes having known mis/nonmatches and mobilities. These reference markers can be chosen to have faster and/or slower mobilities than the test duplex or can provide a ladder of graded mobilities. The position to which the test duplex migrates can then be compared to the reference markers and the position to which the test duplex migrates accurately assigned. The identity of the nucleic acid of interest in the test duplex can then be determined.

The identity of the test duplex or the nucleic acid strands of the test duplex can be determined after separation. In one embodiment, the identity of the nucleic acid of interest is determined based on the mobility assigned to the test duplex compared to the reference markers. The assigned mobility of the test duplex can then be matched to a known nucleic acid to determine the exact identity, and thus sequence, of the nucleic acid of interest. The migration values of known nucleic acids can be compiled into database of values and stored in a computer readable format for ease of analysis and comparison. Examples of such databases are known and can be found for RSCA at www.pel-freez.com. Alternatively, the identity of the nucleic acid of interest can be determined through any of a number of known techniques, including direct sequencing analysis, mass spectrometry and the like. In some of these embodiments, the separated test duplex can be denatured and the individual nucleic acid strands can be tested or identified alone. In some embodiments of the present invention the separation, detection and identification steps can be performed in real time.

Specific examples of separation techniques suitable for use in conjunction with the present invention can be found in PCT application WO 97/20070; Arguello, et al., Reviews in Immunogenetics, 1:209 (1999); Arguello, et al., Bone Marrow Transplantation, 22:527 (1998); Ramon, et al., Human Immunology, 59:734 (1998); Arguello, et al. Tissue Antigen, 52:57 (1998); Madrigal, et al. Immunol. Reviews, 157:153 (1997); Arguello et al., Nature Genetics, 18:192 (1998); Madrigal, et al., Blood Reviews, 11:105 (1997); Arguello, et al., Proc. Nat. Acad. Sci. USA, 93:10961 (1996); Zimmerman et al., Nucleic Acids Research, Vol. 21, No. 19, 4541 (1993); Ramon et al. Hum Immunol. 2002 October; 63(10 Suppl):S98 and Larsen et al. Hum Immunol. 2002 October; 63(10 Suppl):S8 and U.S. Pat. No. 5,750,335. A preferred separation method for use in the present invention is the RSCA® (Reference Strand mediated Conformation Analysis) technique available from Pel-Freez® Clinical Systems. The present invention is suitable for use with any or all of the steps disclosed above or in these references.

Often there is a delicate balance between duplexes which are stable, those that provide consistent pairing and reproducible elution or migration profiles, and duplexes which are unstable, those that have a sufficient number of mismatched/unpaired bases so that elution or migration profiles are not readily repeated. The present inventors have determined that extensive sequence divergence, generally 15% or more, hinders the ability of the reference strand to anneal with the target strand, resulting in the absence of the heteroduplex and the detection of only the homoduplex and the single stranded DNA reference. While it is possible to alter the salt conditions and temperature of annealing and gel conditions, these modification have proven unsatisfactory. As understood by those skilled in the art, unstable duplexes fall somewhere between stable duplexes and nucleic acids that do not readily hybridize together because the unstable duplexes still hybridize together to at least some degree. Of course one skilled in the art will recognize that the hybridization between nucleic acids is a function of the stringency of the conditions in which they are interacted and the present invention accordingly contemplates various degrees of stringency ranging from high to medium to low stringency conditions. As used herein, nucleic acids that have substantially complementary sequences are defined to mean those that form stable duplexes upon hybridization.

As is understood by those skilled in the art, highly stringent conditions generally allow hybridization of highly complementary nucleic acids and describe conditions of low ionic strength and high temperature for washing. Low stringency refers to hybridization conditions having high ionic strength and lower temperature that allow nucleic acids which are less complementary in sequence to hybridize to form a duplex. Variables affecting stringency include, for example, temperature, salt concentration, probe/sample homology and wash conditions. Stringency is increased with a rise in hybridization temperature, all else being equal. Increased stringency provides reduced non-specific hybridization. i.e., less background noise. “High stringency conditions” and “moderate stringency conditions” for nucleic acid hybridization are explained in Current Protocols in Molecular Biology, Ausubel et al., 1998, Green Publishing Associates and Wiley Interscience, NY, the teachings of which are hereby incorporated by reference. Of course, the artisan will appreciate that the stringency of the hybridization conditions can be varied as desired, in order to include or exclude varying degrees of complementation between probe and analyte, in order to achieve the required scope of detection.

Striking a balance between stability and instability in duplexes is particularly problematic when dealing with polymorphic nucleic acids because of the inherent variation in the sequences of such nucleic acids. Thus it is difficult to choose a nucleic acid to hybridize with a polymorphic nucleic acid to form a stable duplex without knowing the sequence of the polymorphic nucleic acid to a large degree. This is particularly true with highly polymorphic nucleic acids, and especially those that are closely genetically related.

One such area where this problem is frequently encountered is in the typing or identification of a tissue sample. Accordingly, preferred methods focus on identifying HLA alleles. The alleles of the HLA loci are classified as Class I—HLA-A, HLA-B, HLA-C, HLA-E, HLA-F and HLA-G, or Class II—HLA-DRA, HLA-DRB1, HLA-DRB2-9, HLA-DQA1, HLA-DQB1, HLA-DPA1, HLA-DPB1, HLA-DMA, HLA-DMB, HLA-DOA and HLA-DOB. There are over a hundred identified alleles that fall in some of these loci and these alleles are closely related and can differ in sequence by only one, or a few, positions. The HLA gene is discussed by Schreuder et al. in Tissue Antigens, 58:109 (2001) and the references disclosed therein, all of which are incorporated by reference. Additional information regarding HLA alleles, and in particular sequence information is available at www.ebi.ac.uk/imgt/hla and www.anthonynolan.org.uk/research.html.

Surprisingly and unexpectedly, it has been discovered that the present methods using universal bases can readily achieve separation and identification of these and other polymorphic nucleic acids with a small number of nucleic acids. This is particularly true for highly polymorphic nucleic acids that are closely related that are not easily subjected to standard duplex analysis. The present methods can involve the methods, techniques and conditions described above.

The present methods allow for the separation and identification of a polymorphic nucleic acid, typically found in a mixture of nucleic acids, that can be related to one another, i.e., they have similar sequences, or not. In some embodiments, the present methods involve hybridizing two nucleic acids, normally in single-stranded form and of the same length, that have roughly complementary sequences with one another and are capable of forming a test duplex. However, the nucleic acids need not be the same length in order for the present methods to be effective and can vary in length, for example by about 1 to about 20 nucleic acids. In fact, additional bases can be added to one of the sequences, either within the nucleic acids or at either terminus, in order to enhance the separation. The test duplex is often a heteroduplex because the sequences of the hybridized nucleic acids are not perfectly complementary, although the present methods can also utilize homoduplexes. As used herein a homoduplex refers nucleic acids which are perfectly complementary in sequence inclusive of nucleic acids hybridized to universal bases. One of the nucleic acids is a reference strand which generally has a known sequence and the other nucleic acid is either a polymorphic nucleic acid or a representation thereof. In these methods, one of the nucleic acids has one or more universal nucleotide bases in a position which corresponds, or is complementary, to one or more positions which are polymorphic in the naturally occurring nucleic acid sequence. In some embodiments, all of the nucleic acid strands that form the test duplex can have one or more universal bases which can be complementary to other universal bases or standard bases on the complementary strand. Universal bases can also be included at positions which are not polymorphic. Once hybridized, the test duplex is separated, such as from other duplexes that are present. Optionally, the separated test duplex can be detected and/or identified. This identification can provide the identity or sequence of the first nucleic acid, the reference nucleic acid, or both.

In some embodiments the test duplex is separated from one or more control duplexes run in the same separation and the positions to which all of the duplexes migrate are detected. The identity of the non-reference strand of the test duplex can then be determined by the position to which the test duplex migrates in the separation. Preferably the position to which the test duplex migrates is assigned based on the positions to which the control duplex(es) migrate.

According to the present methods, identification can be used to type a nucleic acid sequence, i.e., determine the relatedness of one nucleic acid to another, and/or unambiguously identify the sequence of a nucleic acid. Preferably, the nucleic acid of the present invention is DNA, however, one of ordinary skill in the art will recognize that other nucleic acids, including RNA, can be suitably used in the present methods. When typing of a nucleic acid is desired, the reference strand can be obtained from, or represent, a nucleic acid from a specific individual, for example a putative tissue donor or recipient. When the relative relatedness of nucleic acids is determined it is often desirable to use naturally occurring homo- and heteroduplexes related to nucleic acid of interest as control duplexes, such as the homoduplexes found in the prospective tissue donor or recipient. In this embodiment, the sequence of the reference strand can be determined after duplex analysis, or need not be determined at all. One technique for determining genetic relatedness between nucleic acids suitable for use in conjunction with the present invention is disclosed in PCT application WO 95/01453.

When unambiguous identification of a nucleic acid is desired, the position to which the test duplex and one or more control duplexes migrate are determined with a high degree of accuracy. A migration value can then be assigned to the test duplex based on the position of the control duplexes. This migration value can then be compared to compilations of migration values for other known duplexes, preferably located on a computer readable medium in the form of a database. A match in the migration value between the test duplex and a duplex having a known value, where the reference nucleic acid is the same in both, can provide an unambiguous identification of the non-reference strand in the test duplex. Where migration values overlap for duplexes, the separation can be repeated one or more time using the same non-reference strand hybridized to different reference strands to resolve the ambiguity.

In other embodiments where the identity or sequence of a nucleic acid is desired, the nucleic acids of the separated duplex can be directly sequenced, identified using sequence specific probes, such as sequence specific oligonucleotide probes, or using other techniques such as mass spectrometry. The method used to identify the sequence of the nucleic acid is not particularly limited. Sequencing techniques are well known to the skilled artisan and include those found in U.S. Pat. Nos. 4,863,849, 5,405,746, 6,210,891, and 6,258,568 and PCT applications WO 98/13523, WO 98/28440, WO 00/43540, WO 01/42496, WO 02/20836 and WO 02/20837. Such identification can occur in real time or performed sequentially as desired.

As used herein, universal nucleotide, base, nucleoside or the like, refers to a molecule can bind to two or more, i.e., 3, 4, or all 5, naturally occurring bases in a relatively indiscriminate or non-preferential manner. Preferably, the universal base can bind to all of the naturally occurring bases in this manner, such as 2′-deoxyinosine (inosine). Most, preferably, the universal base can bind all of the naturally occurring bases with equal affinity, such as 3-nitropyrrole 2′-deoxynucleoside (3-nitropyrrole) and those disclosed in U.S. Pat. Nos. 5,438,131 and 5,681,947. Generally, when the base is “universal” for only a subset of the natural bases, that subset will generally either be purines (adenine or guanine) or pyrimidines (cytosine, thymine or uracil). Examples of nucleotides that can be considered universal for purines are known as the “K” base (N6-methoxy-2,6-diaminopurine), as discussed in Bergstrom et al., Nucleic Acids Res. 25:1935 (1997) and pyrimidines are known as the “P” base (6H,8H-3,4-dihydropyrimido[4,5-c][1,2]oxazin-7-one), as discussed in Bergstrom et al., supra, and U.S. Pat. No. 6,313,286. Other suitable universal nucleotides include 5-nitroindole (5-nitroindole 2′-deoxynucleoside), 4-nitroindole (4-nitroindole 2′-deoxynucleoside), 6-nitroindole (6-nitroindole 2′-deoxynucleoside) or 2′-deoxynebularine. A partial order of duplex stability has been found as follows: 5-nitroindole>4-nitroindole>6-nitroindole>3-nitropyrrole. Combinations of these universal bases can also be used as desired.

In the present methods, polymorphic nucleic acid is used in its broadest sense, that is it may exist in more than one form. For example, a nucleic acid molecule is said to be polymorphic if it may have more than one specific nucleotide sequence (such as degenerate nucleic acid molecules or genes that may each encode the same protein). A nucleic acid molecule can also be said to be polymorphic if it displays size differences (i.e., differences in length), particularly when comparisons of nucleic acid molecules from different individuals are made.

In one aspect of the present methods, the reference nucleic acid is hybridized with a nucleic acid which is a representation of a polymorphic nucleic acid. Generally, the polymorphic nucleic acid is naturally occurring and is obtained form an individual of interest, such as a putative tissue donor or recipient. The nucleic acid which is the representation of the polymorphic nucleic acid can have the same sequence as the polymorphic nucleic acid but with one or more of the polymorphic positions replaced with one or more universal bases. Alternatively, the nucleic acid that is a representation of the polymorphic nucleic acid can have a sequence that is complementary to the polymorphic sequence and the universal bases are in positions complementary to one or more polymorphic positions of the polymorphic nucleic acid. According to this aspect of the invention, the nucleic acid which is a representation of the polymorphic nucleic acid can be obtained by hybridizing a primer to the polymorphic nucleic acid, or its complementary strand, and extending the primer as desired to obtain a polymorphic nucleic acid that has the sequence complementary to the polymorphic nucleic acid, or the sequence of the polymorphic nucleic acid, respectively. Preferably, the primer itself contains at least some, if not all, of the universal nucleotides which correspond to the polymorphic positions of the nucleic acid because such a reaction is easier to control. However, the present invention also contemplates that some of the universal nucleotides can be incorporated into the nucleic acid during extension of the primer. One method for preferentially adding such a universal nucleotide to a polymorphic position can be performed by the methods disclosed in U.S. Pat. Nos. 6,210,891 and 6,258,568. Alternatively, the entire sequence of the nucleic acid that represents the polymorphic nucleic acid can be produced, such as through synthesis schemes well known in the art. One skilled in the art will realize that supplying a nucleic acid that represents the entire sequence of the polymorphic nucleic acid presupposes a general knowledge of the sequence of the polymorphic nucleic acid. A skilled artisan will also readily understand that this can be easily achieved by focusing the present methods on a desired nucleic acid or DNA target, such as an HLA allele.

In a similar aspect of the present methods, a polymorphic nucleic acid, not a representation thereof, can be used directly in the identification technique. In this aspect, the reference nucleic acid has one or more universal nucleotides placed opposite one or more positions which are known, or are believed to be, polymorphic. In this aspect of the invention, a nucleic acid which is complementary to the polymorphic sequence of interest can also be used. As above, the reference nucleic acid can be introduced into the hybridization reaction as a complete nucleic acid, or the reference nucleic acid can be produced by hybridizing a primer to the polymorphic nucleic acid and elongating the primer in the manner described above.

Surprisingly, it has been found that performing separation and/or identifications according to the present methods, the stability and resolution of the duplexes can be increased and superior separation and identification of polymorphic nucleic acids can result. Without limiting the scope of the present invention it appears that the universal bases in effect reduce the complexity or polymorphicity of the polymorphic nucleic acids thus reducing the complexity of the nucleic acid interactions.

The present invention also provides a single primer, or a primer set made up of a small number of defined primers (such as 2, 3, 4, 5 or more), that is capable of amplifying all or most of the alleles of a polymorphic nucleic acid, for example a polymorphic gene subfamily, such as the alleles of a Class I or Class II HLA loci, and has one or more universal bases at one or more polymorphic positions of the polymorphic nucleic acid. Examples of HLA alleles of the include those classified in Class I—HLA-A, HLA-B, HLA-C, HLA-E, HLA-F and HLA-G, or Class II—HLA-DRA, HLA-DRB1, HLA-DRB2-9, HLA-DQA1, HLA-DQB1, HLA-DPA1, HLA-DPB1, HLA-DMA, HLA-DMB, HLA-DOA and HLA-DOB. And those classified in class II. Preferably, the primers can amplify the alleles of the gene subfamily without amplifying alleles of related subfamilies. These primers are preferably used in a multiplex amplification reaction. Preferably, the minimum necessary amount of positions are replaced with universal bases so that meaningful separation, and thus identification, of the test duplex can be achieved. The number of positions replaced with universal bases will vary depending on several factors, including length and amount of polymorphism present in the nucleic acid. Thus the primer or primer set can be designed that is “universal” for a subset of related nucleic acids, but still provide nucleic acids that can be separated and identified separately by duplex analysis because not all of the polymorphic positions are replaced or hybridized with universal bases. Examples of such primers for HLA DRB1 alleles include the following: SEQ ID NO: 1 (TTGNNGCAGGTTAANNNTGAG), SEQ ID NO: 2 (GNCCCCNCAGCACGTTTCCTGNN GCAGGTTAANNNTGAGTGTCATTTC), SEQ ID NO: 3 (GNCCCCNCAGCACGTTTCNTGNNGNANNNTANNNNNNANTGTNANTTCTT CAAT), SEQ ID NO: 4 (GNCCCCNCAGCACGTTTCNTGNNGNANNNTANNNNTNAG TGTNATTTCTTCAAT) or a nucleic acid having a sequence complementary to SEQ ID NO: 1, 003.343546.1 SEQ ID NO: 2, SEQ ID NO: 3 or SEQ ID NO: 4, wherein N denotes a universal base that is capable of hybridizing to any other nucleic acid residue.

In a preferred embodiment, the nucleic acids that contain universal bases as discussed above are used to stabilize duplex formation of nucleic acids that would otherwise be unstable without the presence of the universal bases. The present inventors have discovered that DNA molecules whose complementarity varies by about 15% or often do not form stable heteroduplexes. The present methods overcome this problem by reducing the sequence divergence between DNA molecules to promote annealing for heteroduplex formation. Sequence divergence is reduced by replacing non-complementary positions with one or more universal bases in a probe, typically scattered throughout the sequence. Accordingly, the present methods are able return the sequence divergence below about 15% so that stable heteroduplexes can be formed. In some embodiments, the sequence divergence is reduced to 14%, 13%, 12%, 11%, 10%, 8%, 6%, 5%, 4%, 3%, 2% or less. To reduce sequence divergence, one or more polymorphic positions, or positions complementary thereto, can be replaced with a universal base. For example, universal bases can replace 1, 2, 3, 4, 5, 6, 7, 8, 9, 10, 15, 20 or more positions depending upon the specific polymorphic nucleic acid. The positions of the universal bases are carefully selected such that significant nucleotide mismatches still remain, facilitating DNA fingerprinting of the target region of interest. Accordingly, sequence ambiguity can be removed from non-target DNA regions, or regions whose sequence is not of interest. As such, duplexes not previously susceptible to duplex separation and identification according to the disclosed techniques can be separated and identified according to the present methods. The present methods and nucleic acids containing universal bases are also advantageous because the number of different nucleic acids needed to carry out different duplex reactions can be reduced, sometimes significantly. For example, a nucleic acid, preferably a primer, can be designed that hybridizes with roughly equal affinity to several different nucleic acids that have different combinations of polymorphisms in the annealing region and thus all of the nucleic acids can be amplified simultaneously without resort to multiple primers. This is particularly useful where the polymorphism(s) are shared in one or more common position between the nucleic acids. In this instance, using a nucleic acid that has a universal base corresponding to the common position(s) of the polymorphic nucleic acids allows for hybridization of the nucleic acid with universal bases to several related polymorphic nucleic acids. The present invention also provides for these primers independent from the method.

The present invention also provides kits for carrying out the methods described herein. In one embodiment, the kit is made up of instructions for carrying out any of the methods described herein. The instructions can be provided in any intelligible form through a tangible medium, such as printed on paper, computer readable media, or the like. The present kits can also include one or more reagents, gels, gelling agents, buffers, hybridization media, nucleic acids, nucleic acid primers, universal bases, molecular weight markers, antibodies, chromatic or fluorescent dyes for staining or labeling a specific targets, radioactive isotopes for labeling specific targets, solid supports and/or disposable lab equipment, such as multi-well plates in order to readily facilitate implementation of the present methods. Solid supports can include beads and the like whereas molecule weight markers can include conjugatable markers, for example biotin and streptavidin or the like. Gelling agents can include agarose or polyacrylamide. Suitable kit components can be found in the description above and the examples below.

### EXAMPLES

### Example 1

Reference Strand mediated Conformation Analysis (RSCA) is based on the PAGE mobility differences among heteroduplexes formed between a labeled reference strand and PCR amplified sample product. The main difficulty for typing HLA-DRB1 was to identify a single primer set to amplify all DRB1 alleles but not other DRB alleles. Furthermore, all amplicons should be the same length. To overcome these difficulties, a multiplex amplification reaction is provided containing group specific forward primers located in the first variable region of Exon II and reverse primers located in Intron II. The high polymorphism present in this exon resulted in unstable heteroduplexes using some reference DNAs. Therefore, the amplification was separated into two reactions. In tube 1 there is a single set of primers specific for DRB1*03/08/11/12/13/14 groups (DRB1*03011, DRB1*0801, DRB1*0821, DRB1*11011, DRB1*1105, DRB1*12011, DRB1*13011, DRB1*1317, DRB1*14011, DRB1*1404, DRB1*1405, DRB1*1439). The primers in tube 2 amplify the remaining groups, DRB1*01/04/07/09/10/14/15 (DRB1*0101,DRB1*0107, DRB1*07011, DRB1*07012, DRB1*15011, DRB1*10011, DRB1*09012, DRB1*04011, DRB1*0434, DRB1*1122, DRB1*1410.). Primers for such amplification are available from Pel-Freez Clinical Systems, LLC. as RSCA kits, including the B-locus kit and multi-dye B-locus kit.

Suitable primers including universal bases that can be used to amplify these alleles include:

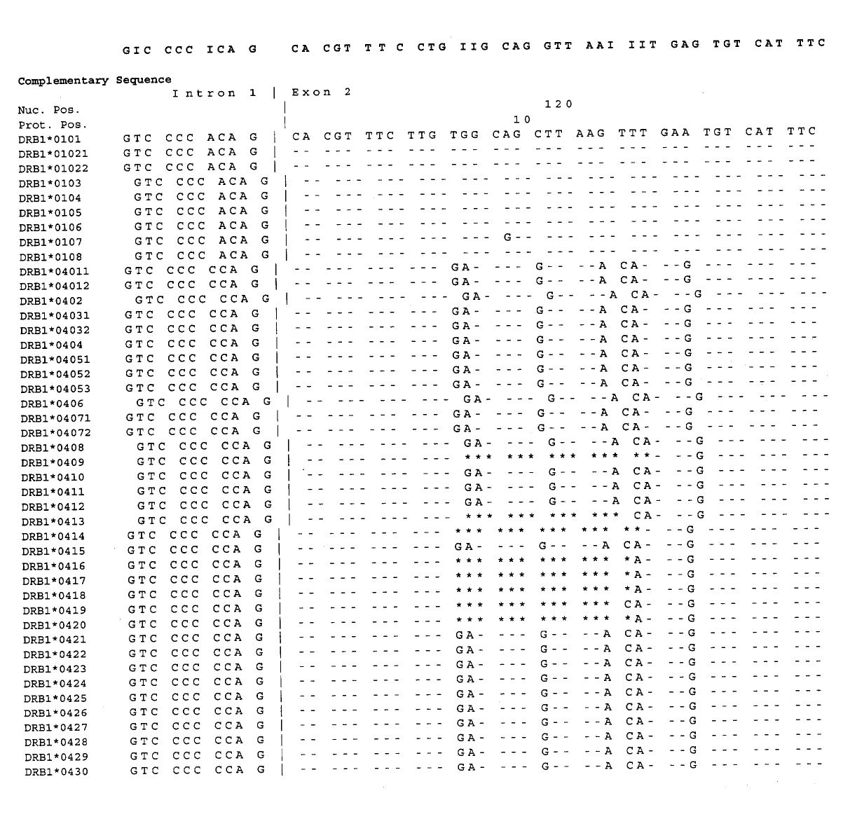

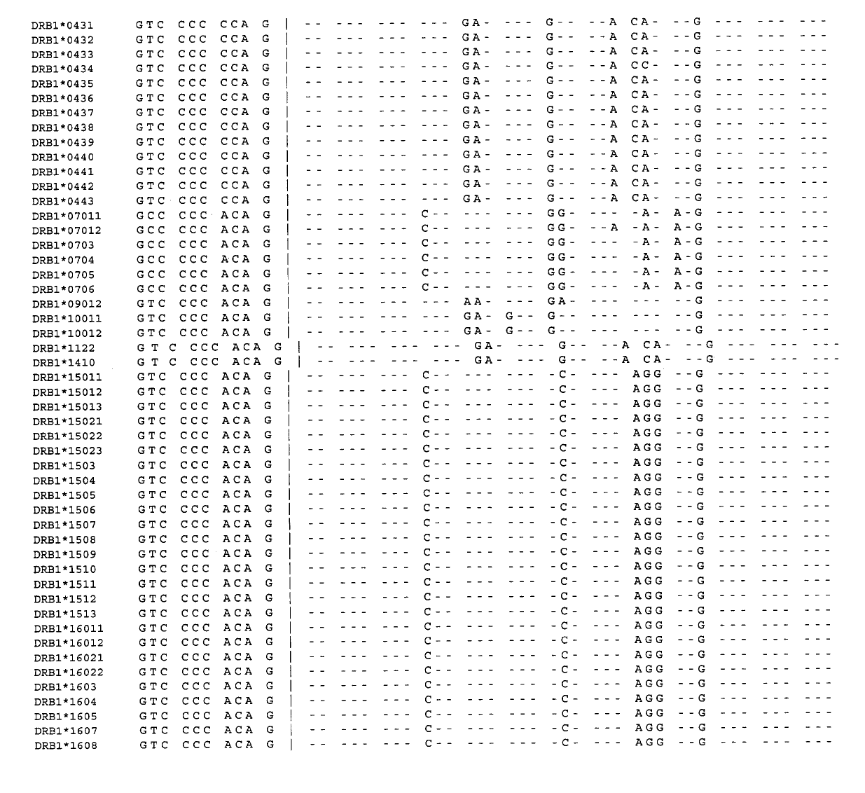

GIC CCC ICA GCA CGT TTC CTG IIG CAG GTT AAI IIT GAG TGT CAT TTC; FIG. 1 illustrates the HLA alleles which this primer can amplify.

Other primers that can be used in conjunction with this primer or alone include:

TTG IIG CAG GTT AAI IIT GAG

GIC CCC ICA GCA CGT TTC ITG IIG IAI IIT AII III IAI TGT IAI TTC TTC AAT; and/or

GIC CCC ICA GCA CGT TTC ITG IIG IAI IIT AII IIT IAG TGT IAT TTC TTC AAT

where I is inosine. Preferably, the primers are labeled at their 5′ end. The above primers including universal bases are capable of amplifying the specifies HLA DRB1 alleles.

DNA samples can be obtained form an individual having a nucleic acid of interest or from well-characterized, serotyped reference samples, such as from UCLA Reference DNA Panel samples. The DNA sample is added to the primer mixtures and amplified, such as on a ALFexpress or ABI377 PRISM®, according to the manufacturer's instructions. Amplified products are then combined with reference DNAs, generally of known sequence, and allowed to hybridize. The duplexes resulting from the hybridization, both homo and hetero, are then separated from one another and other reagents in the reaction mixture. Suitable separation techniques include non-denaturing gel electrophoresis, HPLC or DHPLC. The identity of the nucleic acids in the duplex are then identified. Identification can be performed by assigning a relative migration value, based on reference DNA molecules having known mobility values. This value can then be matched with known migration values for the combination of reference DNA duplexed with other DNAs thereby unambiguously identifying the tested DNA molecule. Examples of suitable kits and protocols for performing this example include those found at www.pel-freez.com for RSCA.

The heteroduplexes for all groups can be separated by the same non-denaturing PAGE at 40° C. The same PCR and heteroduplex annealing conditions are used for RSCA performed on either the ALFexpress or the ABI377 PRISM®. Over 200 IHW and UCLA DNA Exchange samples covering 86 different DRB1 alleles, which represent 99.9% of all known Caucasian alleles, have been typed using this method. Unique mobility values have been assigned for all tested alleles. Among them, 198 (99%) samples are consistent with the previously typed HLA-DRB1 alleles not using universal bases. Two samples, which were typed as DRB1*1501 and DRB1*1201 by SBT, exhibited different mobilities from DRB1*1501 and DRB1*1201 consensus values. These results demonstrate the advantage of RSCA in allele-level typing of DRB1 with minimal ambiguities. The selection of specific primers in the first polymorphic position in Exon II avoids co-amplification of HLA-DRB3, 4 & 5 alleles and, consequently, allows more straightforward interpretation of typing results. Comparing with SBT, the present methods have improved the throughput by 50% using ALFexpress and 25% using ABI 377.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” “more than” and the like include the number recited and refer to ranges which can be subsequently broken down into subranges as discussed above. In the same manner, all ratios disclosed herein also include all subratios falling within the broader ratio.

The present methods can be carried out by performing any of the steps described herein, either alone or in various combinations. Additionally, one skilled in the art will realize that the present invention also encompasses variations of the present methods that specifically exclude one or more of the steps described above.

One skilled in the art will also readily recognize that where members are grouped together in a common manner, such as in a Markush group, the present invention encompasses not only the entire group listed as a whole, but each member of the group individually and all possible subgroups of the main group. Accordingly, for all purposes, the present invention encompasses not only the main group, but also the main group absent one or more of the group members. The present invention also envisages the explicit exclusion of one or more of any of the group members in the claimed invention.

All references disclosed herein are specifically incorporated herein by reference thereto.

While preferred embodiments have been illustrated and described, it should be understood that changes and modifications can be made therein in accordance with ordinary skill in the art without departing from the invention in its broader aspects as defined in the following claims.

What is claimed is:

1. A method for identifying a nucleic acid molecule, comprising:
(a) hybridizing a first nucleic acid whose sequence matches, or is complementary to, a nucleic acid having a plurality of polymorphic positions with a reference nucleic acid whose sequence is substantially complementary to the first nucleic acid to form a test duplex, wherein one or more of the polymorphic positions, or the positions complementary thereto, of the first nucleic acid are replaced with a universal base; and
(b) separating the test duplex.

2. The method of claim 1, further comprising:
(c) detecting the presence or absence of the test duplex;
(d) identifying the first nucleic acid; or
(e) both (c) and (d).

3. The method of claim 1 wherein the first nucleic acid is produced by:
(i) hybridizing a polymorphic nucleic acid having one or more polymorphic positions or a nucleic acid complementary thereto with a complementary nucleic acid primer that has at least one universal base complementary to at least one polymorphic position of the polymorphic nucleic acid; and
(ii) elongating the nucleic acid primer.

4. The method of claim 1 wherein the first nucleic acid is an HLA allele.

5. The method of claim 4 wherein the HLA allele is HLA-DRB1.

6. The method of claim 5 wherein all of the different first nucleic acids have at least one universal base in a polymorphic position, or position complementary thereto, which is common to all of the HLA-DRB1 alleles.

7. The method of claim 1 wherein the universal base is 2′-deoxyinosine, 3-nitropyrrole (3-nitropyrrole 2′-deoxynucleoside), 5-nitroindole (5-nitroindole 2′-deoxynucleoside), 4-nitroindole (4-nitroindole 2′-deoxynucleoside), 6-nitroindole (6-nitroindole 2′-deoxynucleoside), 2′-deoxynebularine, N6-methoxy-2,6-diaminopurine, or 6H,8H-3,4-dihydropyrimido[4,5-c][1,2]oxazin-7-one.

8. The method of claim 1 wherein the first nucleic acid, reference nucleic acid or both are labeled.

9. A method for identifying a nucleic acid molecule, comprising:
(a) hybridizing a polymorphic nucleic acid having one or more polymorphic positions with a reference nucleic acid that is substantially complementary to a reference nucleic acid to form a test duplex, wherein one or more positions of the reference nucleic acid complementary to the one or more polymorphic position are replaced with a universal base; and
(b) separating the test duplex.

10. The method of claim 9 further comprising:
(c) detecting the presence or absence of the test duplex;
(d) identifying the polymorphic nucleic acid; or
(e) both (c) and (d).

11. The method of claim 9 wherein the polymorphic nucleic acid does not form a stable duplex with the reference nucleic acid in the absence of the nucleic acid having universal bases.

12. The method of claim 9 wherein the reference nucleic acid has a sufficient amount of the positions complementary to the one or more polymorphic positions of the polymorphic nucleic acid replaced with universal bases in order to stabilize the test duplex relative to a test duplex with a control nucleic acid having the same sequence that does not have universal nucleic acids and further wherein a sufficient amount of the polymorphic positions, or positions complementary thereto, are not replaced with universal bases such that all alleles which have different polymorphic sequences within the primer annealing region can be identified utilizing the same reference nucleic acid.

13. The method of claim 9 wherein the polymorphic nucleic acid is an HLA allele.

14. The method of claim 13 wherein the reference nucleic acid is capable of specifically identifying all of the HLA alleles of an HLA gene subfamily but not the HLA alleles of related HLA gene subfamilies.

15. The method of claim 14 wherein the HLA gene subfamily is HLA-DRB1.

16. The method of claim 15 wherein the reference nucleic acid comprises the sequence set forth in SEQ ID NO: 1 (TTGNNGCAGGTTAANNNTGAG), SEQ ID NO: 2 (GNCCCCNCAGCACGTTTCCTGNN GCAGGTTAANNNTGAGTGTCATTTC), SEQ ID NO: 3 (GNCCCCNCAGCACGTTTCNTGNNGNANNNTANNNNNNANTGTNANTTCTTCAAT), SEQ ID NO: 4 (GNCCCCNCAGCACGTTTCNTGNNGNANNNTANNNNTNAGTGTNATTTCTTCAAT) or a nucleic acid having a sequence complementary to SEQ ID NO: 1, SEQ ID NO: 2, SEQ ID NO: 3 or SEQ ID NO:4.

17. The method of claim 9 wherein the polymorphic nucleic acid, reference nucleic acid or both are labeled.

18. A modified nucleic acid comprising the sequence set forth in SEQ ID NO: 1 (TTGNNGCAGGTTAANNNTGAG), SEQ ID NO: 2 (GNCCCCNCAGCACGTTTCCTGNN GCAGGTTAANNNTGAGTGTCATTTC), SEQ ID NO: 3 (GNCCCCNCAGCACGTTTCN TGNNGNANNNTANNNNNNANTGTNANTTCTTCAAT), SEQ ID NO: 4 (GNCCC CNCAGCACGTTTCNTGNNGNANNNTANNNNTNAGTGTNATTTCTTCAAT) or a nucleic acid having a sequence complementary to SEQ ID NO: 1, SEQ ID NO: 2, SEQ ID NO: 3 or SEQ ID NO: 4, wherein N denotes a universal base that is capable of hybridizing to any other nucleic acid residue.

19. The modified nucleic acid of claim 20 wherein the universal base is 2′-deoxyinosine, 3-nitropyrrole (3-nitropyrrole 2′-deoxynucleoside), 5-nitroindole (5-nitroindole 2′-deoxynucleoside), 4-nitroindole (4-nitroindole 2′-deoxynucleoside), 6-nitroindole (6-nitroindole 2′-deoxynucleoside), 2′-deoxynebularine, N6-methoxy-2,6-diaminopurine, or 6H,8H-3,4-dihydropyrimido[4,5-c][1,2]oxazin-7-one.

20. A kit for identifying a nucleic acid molecule comprising: (i) instructions for carrying out the method of claim 1 and one or more: (ii) reagents, (iii) primers, (iv) solid supports (v) enzymes or (vi) pieces of lab equipment.

21. A kit for identifying a nucleic acid molecule comprising: (i) instructions for carrying out the method of claim 9 and one or more: (ii) reagents, (iii) primers, (iv) solid supports (v) enzymes or (vi) pieces of lab equipment.


SEQUENCE LISTING (101 sequences)

SEQ ID NO: 1 - DNA, 21 bp, Artificial Sequence
  synthetic nucleic acid
ttgnngcaggttaannntgag

SEQ ID NO: 2 - DNA, 48 bp, Artificial Sequence
  synthetic nucleic acid
gnccccncagcacgtttcctgnngcaggttaannntgagtgtcatttc

SEQ ID NO: 3 - DNA, 54 bp, Artificial Sequence
  synthetic nucleic acid
gnccccncagcacgtttcntgnngnannntannnnnnantgtnanttcttcaat

SEQ ID NO: 4 - DNA, 54 bp, Artificial Sequence
  synthetic nucleic acid
gnccccncagcacgtttcntgnngnannntannnntnagtgtnatttcttcaat

SEQ ID NO: 5 - DNA, 48 bp, Artificial Sequence; identical to SEQ ID NO: 2
  primer

SEQ ID NO: 6 - DNA, 75 bp, Artificial Sequence
  primer
ttgnngcaggttaannntgaggnccccncagcacgtttcntgnngnannntannnnnnantgtnanttcttcaat

SEQ ID NO: 7 - DNA, 54 bp, Artificial Sequence; identical to SEQ ID NO: 4
  primer

SEQ ID NO: 8 - DNA, 48 bp, Homo sapiens
gtccccacagcacgtttcttgtggcagcttaagtttgaatgtcatttc

SEQ ID NO: 9 - DNA, 48 bp, Homo sapiens; identical to SEQ ID NO: 8

SEQ ID NO: 10 - DNA, 48 bp, Homo sapiens; identical to SEQ ID NO: 8

SEQ ID NO: 11 - DNA, 48 bp, Homo sapiens; identical to SEQ ID NO: 8

SEQ ID NO: 12 - DNA, 48 bp, Homo sapiens; identical to SEQ ID NO: 8

SEQ ID NO: 13 - DNA, 48 bp, Homo sapiens; identical to SEQ ID NO: 8

SEQ ID NO: 14 - DNA, 48 bp, Homo sapiens; identical to SEQ ID NO: 8

SEQ ID NO: 15 - DNA, 48 bp, Homo sapiens
gtccccacagcacgtttcttgtgggagcttaagtttgaatgtcatttc

SEQ ID NO: 16 - DNA, 48 bp, Homo sapiens; identical to SEQ ID NO: 8

SEQ ID NO: 17 - DNA, 48 bp, Homo sapiens
gtccccccagcacgtttcttggagcaggttaaacatgagtgtcatttc

SEQ ID NO: 18 - DNA, 48 bp, Homo sapiens; identical to SEQ ID NO: 17

SEQ ID NO: 19 - DNA, 48 bp, Homo sapiens; identical to SEQ ID NO: 17

SEQ ID NO: 20 - DNA, 48 bp, Homo sapiens; identical to SEQ ID NO: 17

SEQ ID NO: 21 - DNA, 48 bp, Homo sapiens; identical to SEQ ID NO: 17

SEQ ID NO: 22 - DNA, 48 bp, Homo sapiens; identical to SEQ ID NO: 17

SEQ ID NO: 23 - DNA, 48 bp, Homo sapiens; identical to SEQ ID NO: 17

SEQ ID NO: 24 - DNA, 48 bp, Homo sapiens; identical to SEQ ID NO: 17

SEQ ID NO: 25 - DNA, 48 bp, Homo sapiens; identical to SEQ ID NO: 17

SEQ ID NO: 26 - DNA, 48 bp, Homo sapiens; identical to SEQ ID NO: 17

SEQ ID NO: 27 - DNA, 48 bp, Homo sapiens; identical to SEQ ID NO: 17

SEQ ID NO: 28 - DNA, 48 bp, Homo sapiens; identical to SEQ ID NO: 17

SEQ ID NO: 29 - DNA, 48 bp, Homo sapiens; identical to SEQ ID NO: 17

SEQ ID NO: 30 - DNA, 48 bp, Homo sapiens
  misc_feature (22)..(35): N can be any base
gtccccccagcacgtttcttgnnnnnnnnnnnnnntgagtgtcatttc

SEQ ID NO: 31 - DNA, 48 bp, Homo sapiens; identical to SEQ ID NO: 17

SEQ ID NO: 32 - DNA, 48 bp, Homo sapiens; identical to SEQ ID NO: 17

SEQ ID NO: 33 - DNA, 48 bp, Homo sapiens; identical to SEQ ID NO: 17

SEQ ID NO: 34 - DNA, 48 bp, Homo sapiens
  misc_feature (21)..(33): N can be any base
gtccccccagcacgtttcttgnnnnnnnnnnnncatgagtgtcatttc

SEQ ID NO: 35 - DNA, 48 bp, Homo sapiens; identical to SEQ ID NO: 30
  misc_feature (22)..(35): N can be any base

SEQ ID NO: 36 - DNA, 48 bp, Homo sapiens; identical to SEQ ID NO: 17

SEQ ID NO: 37 - DNA, 48 bp, Homo sapiens
  misc_feature (22)..(34): N can be any base
gtccccccagcacgtttcttgnnnnnnnnnnnnnatgagtgtcatttc

SEQ ID NO: 38 - DNA, 48 bp, Homo sapiens; identical to SEQ ID NO: 37
  misc_feature (22)..(34): N can be any base

SEQ ID NO: 39 - DNA, 48 bp, Homo sapiens; identical to SEQ ID NO: 37
  misc_feature (22)..(34): N can be any base

SEQ ID NO: 40 - DNA, 48 bp, Homo sapiens; identical to SEQ ID NO: 34
  misc_feature (22)..(33): N can be any base

SEQ ID NO: 41 - DNA, 48 bp, Homo sapiens; identical to SEQ ID NO: 37
  misc_feature (22)..(34): N can be any base

SEQ ID NO: 42 - DNA, 48 bp, Homo sapiens; identical to SEQ ID NO: 17

SEQ ID NO: 43 - DNA, 48 bp, Homo sapiens; identical to SEQ ID NO: 17

SEQ ID NO: 44 - DNA, 48 bp, Homo sapiens; identical to SEQ ID NO: 17

SEQ ID NO: 45 - DNA, 48 bp, Homo sapiens; identical to SEQ ID NO: 17

SEQ ID NO: 46 - DNA, 48 bp, Homo sapiens; identical to SEQ ID NO: 17

SEQ ID NO: 47 - DNA, 48 bp, Homo sapiens; identical to SEQ ID NO: 17

SEQ ID NO: 48 - DNA, 48 bp, Homo sapiens; identical to SEQ ID NO: 17

SEQ ID NO: 49 - DNA, 48 bp, Homo sapiens; identical to SEQ ID NO: 17

SEQ ID NO: 50 - DNA, 48 bp, Homo sapiens; identical to SEQ ID NO: 17

SEQ ID NO: 51 - DNA, 48 bp, Homo sapiens; identical to SEQ ID NO: 17

SEQ ID NO: 52 - DNA, 48 bp, Homo sapiens; identical to SEQ ID NO: 17

SEQ ID NO: 53 - DNA, 48 bp, Homo sapiens; identical to SEQ ID NO: 17

SEQ ID NO: 54 - DNA, 48 bp, Homo sapiens; identical to SEQ ID NO: 17

SEQ ID NO: 55 - DNA, 48 bp, Homo sapiens
gtccccccagcacgtttcttggagcaggttaaacctgagtgtcatttc

SEQ ID NO: 56 - DNA, 48 bp, Homo sapiens; identical to SEQ ID NO: 17

SEQ ID NO: 57 - DNA, 48 bp, Homo sapiens; identical to SEQ ID NO: 17

SEQ ID NO: 58 - DNA, 48 bp, Homo sapiens; identical to SEQ ID NO: 17

SEQ ID NO: 59 - DNA, 48 bp, Homo sapiens; identical to SEQ ID NO: 17

SEQ ID NO: 60 - DNA, 48 bp, Homo sapiens; identical to SEQ ID NO: 17

SEQ ID NO: 61 - DNA, 48 bp, Homo sapiens; identical to SEQ ID NO: 17

SEQ ID NO: 62 - DNA, 48 bp, Homo sapiens; identical to SEQ ID NO: 17

SEQ ID NO: 63 - DNA, 48 bp, Homo sapiens; identical to SEQ ID NO: 17

SEQ ID NO: 64 - DNA, 48 bp, Homo sapiens; identical to SEQ ID NO: 17

SEQ ID NO: 65 - DNA, 48 bp, Homo sapiens
gcccccacagcacgtttcctgtggcagggtaagtataagtgtcatttc

SEQ ID NO: 66 - DNA, 48 bp, Homo sapiens
gcccccacagcacgtttcctgtggcagggtaaatataagtgtcatttc

SEQ ID NO: 67 - DNA, 48 bp, Homo sapiens; identical to SEQ ID NO: 65

SEQ ID NO: 68 - DNA, 48 bp, Homo sapiens; identical to SEQ ID NO: 65

SEQ ID NO: 69 - DNA, 48 bp, Homo sapiens; identical to SEQ ID NO: 65

SEQ ID NO: 70 - DNA, 48 bp, Homo sapiens; identical to SEQ ID NO: 65

SEQ ID NO: 71 - DNA, 48 bp, Homo sapiens
gtccccacagcacgtttcttgaagcaggataagtttgagtgtcatttc

SEQ ID NO: 72 - DNA, 48 bp, Homo sapiens
gtccccacagcacgtttcttggaggaggttaagtttgagtgtcatttc

SEQ ID NO: 73 - DNA, 48 bp, Homo sapiens; identical to SEQ ID NO: 72

SEQ ID NO: 74 - DNA, 48 bp, Homo sapiens
gtccccacagcacgtttcttggaggaggttaaacatgagtgtcatttc

SEQ ID NO: 75 - DNA, 48 bp, Homo sapiens; identical to SEQ ID NO: 74

SEQ ID NO: 76 - DNA, 48 bp, Homo sapiens
gtccccacagcacgtttcctgtggcagcctaagagggagtgtcatttc

SEQ ID NO: 77 - DNA, 48 bp, Homo sapiens; identical to SEQ ID NO: 76

SEQ ID NO: 78 - DNA, 48 bp, Homo sapiens; identical to SEQ ID NO: 76

SEQ ID NO: 79 - DNA, 48 bp, Homo sapiens; identical to SEQ ID NO: 76

SEQ ID NO: 80 - DNA, 48 bp, Homo sapiens; identical to SEQ ID NO: 76

SEQ ID NO: 81 - DNA, 48 bp, Homo sapiens; identical to SEQ ID NO: 76

SEQ ID NO: 82 - DNA, 48 bp, Homo sapiens; identical to SEQ ID NO: 76

SEQ ID NO: 83 - DNA, 48 bp, Homo sapiens; identical to SEQ ID NO: 76

SEQ ID NO: 84 - DNA, 48 bp, Homo sapiens; identical to SEQ ID NO: 76

SEQ ID NO: 85 - DNA, 48 bp, Homo sapiens; identical to SEQ ID NO: 76

SEQ ID NO: 86 - DNA, 48 bp, Homo sapiens; identical to SEQ ID NO: 76

SEQ ID NO: 87 - DNA, 48 bp, Homo sapiens; identical to SEQ ID NO: 76

SEQ ID NO: 88 - DNA, 48 bp, Homo sapiens; identical to SEQ ID NO: 76

SEQ ID NO: 89 - DNA, 48 bp, Homo sapiens; identical to SEQ ID NO: 76

SEQ ID NO: 90 - DNA, 48 bp, Homo sapiens; identical to SEQ ID NO: 76

SEQ ID NO: 91 - DNA, 48 bp, Homo sapiens; identical to SEQ ID NO: 76

SEQ ID NO: 92 - DNA, 48 bp, Homo sapiens; identical to SEQ ID NO: 76

SEQ ID NO: 93 - DNA, 48 bp, Homo sapiens; identical to SEQ ID NO: 76

SEQ ID NO: 94 - DNA, 48 bp, Homo sapiens; identical to SEQ ID NO: 76

SEQ ID NO: 95 - DNA, 48 bp, Homo sapiens; identical to SEQ ID NO: 76

SEQ ID NO: 96 - DNA, 48 bp, Homo sapiens; identical to SEQ ID NO: 76

SEQ ID NO: 97 - DNA, 48 bp, Homo sapiens; identical to SEQ ID NO: 76

SEQ ID NO: 98 - DNA, 48 bp, Homo sapiens; identical to SEQ ID NO: 76

SEQ ID NO: 99 - DNA, 48 bp, Homo sapiens; identical to SEQ ID NO: 76

SEQ ID NO: 100 - DNA, 48 bp, Homo sapiens; identical to SEQ ID NO: 76

SEQ ID NO: 101 - DNA, 48 bp, Homo sapiens; identical to SEQ ID NO: 76